(19) United States
(12) Patent Application Publication
(10) Pub. No.: US 2009/0286999 A1
(43) Pub. Date: Nov. 19, 2009

(54) CATALYST SYSTEM FOR PREPARING CARBOXYLIC ACIDS AND/OR CARBOXYLIC ANHYDRIDES

(75) Inventors: Hagen Wilmer, Ludwigshafen (DE); Cornelia Dobner, Ludwigshafen (DE); Frank Rosowski, Mannheim (DE); Jürgen Zühlke, Speyer (DE); Sebastian Storck, Mannheim (DE); Hans-Martin Allmann, Neunkirchen (DE); Thomas Lautensack, Birkenau (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/296,885

(22) PCT Filed: Apr. 5, 2007
(86) PCT No.: PCT/EP07/53376

§ 371 Date: Oct. 10, 2008

(30) Foreign Application Priority Data
Apr. 12, 2006 (EP) 06112610.0

(51) Int. Cl. C07D 307/89; B01J 8/04; C07C 51/21

(52) U.S. Cl. 549/248; 422/196; 562/512.2

(57) ABSTRACT

The present invention relates to a catalyst system for preparing carboxylic acids and/or carboxylic anhydrides which has at least three catalyst layers arranged one on top of the other in the reaction tube, with the proviso that the most inactive catalyst layer is preceded in the upstream direction by a more active catalyst layer. The invention further relates to a process for gas phase oxidation in which a gaseous stream which comprises one hydrocarbon and molecular oxygen is passed through a plurality of catalyst layers, the least active catalyst layer being upstream of a more active catalyst layer.

The present invention relates to a catalyst system for preparing carboxylic acids and/or carboxylic anhydrides which has at least three catalyst layers arranged one on top of the other in the reaction tube, with the proviso that the most inactive catalyst layer is preceded in the upstream direction by a more active catalyst layer. The invention further relates to a process for gas phase oxidation in which a gaseous stream which comprises one hydrocarbon and molecular oxygen is passed through a plurality of catalyst layers, the least active catalyst layer being upstream of a more active catalyst layer.

A multitude of carboxylic acids and/or carboxylic anhydrides is prepared industrially by the catalytic gas phase oxidation of hydrocarbons such as benzene, the xylenes, naphthalene, toluene or durene in fixed bed reactors. In this way, it is possible to obtain, for example, benzoic acid, maleic anhydride, phthalic anhydride, isophthalic acid, terephthalic acid or pyromellitic anhydride. In general, a mixture of an oxygenous gas and the starting material to be oxidized is passed through tubes in which a bed of a catalyst is disposed. For temperature regulation, the tubes are surrounded by a heat carrier medium, for example a salt melt.

Even though the excess heat of reaction is removed by the heat carrier medium, local temperature maxima (hotspots) can be formed in the catalyst bed, in which there is a higher temperature than in the remaining part of the catalyst bed, or in the remaining part of the catalyst layer. These hotspots lead to side reactions, such as the total combustion of the starting material, or to the formation of undesired by-products which can be removed from the reaction product only at great cost and inconvenience, if at all.

Moreover, the catalyst can be damaged irreversibly from a certain hotspot temperature. Therefore, when starting up the process, the loading of the gaseous stream with the hydrocarbon to be oxidized has to be kept very low at first and can be increased only slowly. The final production state is often attained only after a few weeks.

Experience has shown that these catalysts have a lifetime of from 2 to 5 operating years, after which their activity declines, both with respect to the conversion and the selectivity, to such an extent that further use is no longer economically viable.

To attenuate these hotspots, various measures have been taken. In particular, as described in DE-A 40 13 051, there has been a transition to arranging catalysts of different activity layer by layer in the catalyst bed, the less active catalyst generally being disposed toward the gas inlet and the more active catalyst toward the gas outlet.

DE 198 23 262 A describes a process for preparing phthalic anhydride with at least three coated catalysts arranged in layers one on top of the other, the catalyst activity rising from layer to layer from the gas inlet side to the gas outlet side.

EP-A 1 063 222 describes a process for preparing phthalic anhydride which is performed in one or more fixed bed reactors. The catalyst beds in the reactors have three or more than three individual catalyst layers in succession in the reactor. After passing through the first catalyst layer under the reaction conditions, from 30 to 70% by weight of the o-xylene, naphthalene or of the mixture of the two used has been converted. After the second layer, 70% by weight or more has been converted.

WO 2005/115616 describes a process for preparing phthalic anhydride in a fixed bed reactor having three or more catalyst layers with activity increasing in flow direction. It is disclosed that the content of the active compositions and hence the layer thicknesses of the catalysts decreases advantageously in flow direction.

The activity of the catalysts or catalyst systems used for the gas phase oxidation decreases with increasing operating time. A high proportion of unconverted hydrocarbons or partly oxidized intermediates gets into regions of the catalyst bed further downstream. The reaction increasingly shifts toward the reactor outlet and the hotspot migrates downstream. The catalyst deactivation can be counteracted to a certain degree by increasing the temperature of the heat carrier medium. The increase in the temperature of the heat carrier medium and/or the shifting of the hotspot lead, in the case of multilayer catalyst systems, to an increase in the temperature with which the gas mixture enters a downstream catalyst layer. Since downstream catalyst layers are generally more active but less selective, undesired overoxidation and other side reactions increase. The two effects mentioned result in a decrease in the product yield and selectivity with operating time.

In general, accordingly, in spite of the problems indicated, the activity is increased starting from the reactor inlet to the reactor outlet, since the low activity of the first catalyst layers brings about a high selectivity and hence a high yield of desired product.

It is an object of the invention to provide a catalyst system for gas phase oxidation, which has very uniform thermal stress on the catalyst system. It is thus a further object of the invention to provide a catalyst system for gas phase oxidation which forms the initial hotspot very close to the reactor inlet.

The object is achieved by a catalyst system for preparing carboxylic acids and/or carboxylic anhydrides which has at least three catalyst layers arranged one on top of the other in the reaction tube, with the proviso that the most inactive catalyst layer is preceded in the upstream direction by a more active catalyst layer.

A catalyst layer is considered to be the bed of a catalyst with essentially uniform activity, i.e. with essentially uniform composition of the active composition, active composition content and packing density (disregarding unavoidable fluctuations in the filling of the reactor). Successive catalyst layers thus differ in the activity of the catalysts present.

In the present invention, the activity of a catalyst layer is defined as follows: the higher the conversion for a specific reactant mixture at the same salt bath temperature, the higher the activity.

A higher activity of the catalysts can be achieved, for example, by addition, or increased addition, of activity-increasing promoters to the active composition and/or by lower addition of activity-lowering promoters and/or by a higher BET surface area of the catalysts and/or by a higher active composition content, i.e. by a higher active composition per unit volume and/or by increasing the empty space between the individual shaped catalyst bodies and/or by decreasing the content of inert substances. In addition, a higher activity can be increased by a specific pore distribution.

The catalytically active composition of all catalysts preferably comprises at least vanadium oxide and titanium dioxide. Measures for increasing the activity of gas phase oxidation catalysts based on vanadium oxide and titanium dioxide are known per se to those skilled in the art.

For instance, the catalytically active composition may comprise oxidic compounds which, as promoters, influence the activity and selectivity of the catalyst, for example by lowering or increasing its activity.

Examples of activity-influencing promoters include the alkali metal oxides, especially cesium oxide, lithium oxide, potassium oxide and rubidium oxide, thallium(I) oxide, aluminum oxide, zirconium oxide, iron oxide, nickel oxide, cobalt oxide, manganese oxide, tin oxide, silver oxide, copper oxide, chromium oxide, molybdenum oxide, tungsten oxide, iridium oxide, tantalum oxide, niobium oxide, arsenic oxide, antimony oxide, cerium oxide. In general, from this group, cesium is used as the promoter. Useful sources of these elements include the oxides or hydroxides or the salts which can be converted thermally to oxides, such as carboxylates, especially the acetates, malonates or oxalates, carbonates, hydrogencarbonates or nitrates. Oxidic phosphorus compounds, especially phosphorus pentoxide, are also suitable as activity-influencing promoters. Useful phosphorus sources include in particular phosphoric acid, phosphorous acid, hypophosphorous acid, ammonium phosphate or phosphoric esters and in particular ammonium dihydrogen phosphate. Suitable activity-increasing additives also include various antimony oxides, especially antimony trioxide.

A further means of increasing the activity consists in the variation of the content of the active composition in the total weight of the catalyst, higher active composition contents causing a higher activity and vice versa.

Advantageously, the higher activity of the upstream catalyst layer is established by virtue of a lower content of cesium in the active composition, by virtue of a higher active mass per unit tube volume, by virtue of a higher content of vanadium in the active composition, by virtue of a higher BET surface area of the catalysts or by virtue of a combination of these means. The higher activity of the upstream catalyst layer is preferably achieved by virtue of a lower content of cesium or by virtue of a higher active composition per unit tube volume, especially by virtue of a lower content of cesium.

In the case of the increase in activity by virtue of a smaller addition of cesium to the active composition, advantageously from 1 to 50% less cesium, based on the cesium content of the downstream catalyst layer, is used in the upstream catalyst layer. Preference is given to using from 5 to 25% less cesium, based on the cesium content of the downstream catalyst layer, in particular from 10 to 20% less cesium.

In the case of the increase in activity by virtue of an increase in the active composition, advantageously from 105 to 200% of active composition, based on the active composition of the downstream catalyst layer, is used in the upstream catalyst layer. Preference is given to using from 110 to 150% of active composition, based on the active composition of the downstream catalyst layer, in particular from 120 to 130% of active composition.

In the case of the increase in activity by virtue of an increased addition of vanadium to the active composition, advantageously from 105 to 200% of vanadium, based on the vanadium content of the downstream catalyst layer, is used in the upstream catalyst layer. Preference is given to using from 110 to 150% of vanadium, based on the vanadium content of the downstream catalyst layer, in particular from 120 to 130% of vanadium.

In the case of the increase in activity by virtue of an increased BET surface area of the catalyst, the catalyst in the upstream catalyst layer advantageously has a BET surface area increased by from 5 to 100%, based on the BET surface area of the catalyst of the downstream catalyst layer. The catalyst preferably has a BET surface area increased by from 10 to 50%, in particular a BET surface area increased by from 20 to 30%.

When combinations of the activity-structuring methods indicated are used, the suitable combinations and their amount can be determined by the person skilled in the art by a few experiments.

The BET surface area of the catalytically active components of the catalyst is advantageously in the range from 5 to 50 m²/g, preferably from 5 to 40 m²/g, in particular from 9 to 35 m²/g.

The active composition content is preferably from 3 to 15% by weight, in particular from 4 to 12% by weight, based on the total catalyst mass.

The catalysts used in the process according to the present invention are generally coated catalysts in which the catalytically active composition is applied in coating form on an inert support. The layer thickness of the catalytically active composition is generally from 0.02 to 0.25 mm, preferably from 0.05 to 0.15 mm. In general, the catalysts have an active composition layer with essentially homogeneous chemical composition applied in coating form. In addition, it is also possible for two or more different active composition layers to be applied successively to one support. Reference is then made to a two-layer or multilayer catalyst (see, for example, DE 19839001 A1).

The inert carrier material used may be virtually all prior art carrier materials, as find use advantageously in the preparation of coated catalysts for the oxidation of aromatic hydrocarbons to aldehydes, carboxylic acids and/or carboxylic anhydrides, as described, for example, in WO 2004/103561 on pages 5 and 6. Preference is given to using steatite in the form of spheres having a diameter of from 3 to 6 mm or of rings having an external diameter of from 5 to 9 mm, a length of from 4 to 7 mm and an internal diameter of from 3 to 7 mm.

The individual layers of the coated catalyst can be applied by any methods known per se, for example by spraying-on solutions or suspensions in a coating drum or coating with a solution or suspension in a fluidized bed, as described, for example, in WO 2005/030388, DE 4006935 A1, DE 19824532 A1, EP 0966324 B1.

The upstream catalyst layer and the most inactive catalyst layer are followed by at least one further layer, advantageously from two to four further layers, in particular two or three further catalyst layers.

Advantageously, in a four-layer catalyst system, the upstream catalyst layer, based on the total length of the catalyst bed, has from 1 to 40%, preferably from 5 to 25%, in particular from 10 to 20%. The second catalyst layer has advantageously, based on the total length of the catalyst bed, from 15 to 75%, preferably from 25 to 60%, in particular from 30 to 50%. The third catalyst layer has advantageously, based on the total length of the catalyst bed, from 5 to 45%, preferably from 10 to 40%, in particular from 15 to 30%. The fourth catalyst layer likewise has advantageously, based on the total length of the catalyst bed, from 5 to 45%, preferably from 10 to 40%, in particular from 15 to 30%.

Advantageously, in a four-layer catalyst system, the bed length of the upstream catalyst layer is from 5 cm to 120 cm, preferably from 15 cm to 75 cm, in particular from 30 cm to 60 cm, the bed length of the second catalyst layer is from 45 cm to 225 cm, preferably from 75 cm to 180 cm, in particular from 90 cm to 150 cm, the bed length of the third catalyst layer is from 15 cm to 135 cm, preferably from 30 cm to 120 cm, in particular from 45 cm to 90 cm, and the bed length of the fourth catalyst layer is from 15 cm to 135 cm, preferably from 30 cm to 120 cm, in particular from 45 cm to 90 cm.

Advantageously, in a five-layer catalyst system, the upstream catalyst layer, based on the total length of the catalyst bed, has from 1 to 40%, preferably from 5 to 25%, in particular from 10 to 20%. The second catalyst layer has advantageously, based on the total length of the catalyst bed, from 15 to 75%, preferably from 25 to 60%, in particular from 30 to 50%. The third catalyst layer has advantageously, based on the total length of the catalyst bed, from 5 to 45%, preferably from 5 to 30%, in particular from 10 to 20%. The fourth catalyst layer has advantageously, based on the total length of the catalyst bed, from 5 to 45%, preferably from 5 to 30%, in particular from 10 to 25%. The fifth catalyst layer likewise has advantageously, based on the total length of the catalyst bed, from 5 to 45%, preferably from 5 to 30%, in particular from 10 to 25%.

Advantageously, in a five-layer catalyst system, the bed length of the upstream catalyst layer is from 5 cm to 120 cm, preferably from 15 cm to 75 cm, in particular from 30 cm to 60 cm, the bed length of the second catalyst layer is from 45 cm to 225 cm, preferably from 75 cm to 180 cm, in particular from 90 cm to 150 cm, the bed length of the third catalyst layer is from 15 cm to 135 cm, preferably from 15 cm to 90 cm, in particular from 30 cm to 60 cm, and the bed length of the fourth catalyst layer is from 15 cm to 135 cm, preferably from 15 cm to 90 cm, in particular from 30 cm to 75 cm, and the bed length of the fifth catalyst layer is from 15 cm to 135 cm, preferably from 15 cm to 90 cm, in particular from 30 cm to 75 cm.

The upstream catalyst layer accordingly advantageously makes up from 1 to 40 percent of the total bed length of the catalyst system, preferably from 5 to 25%, in particular from 10 to 20 percent.

Advantageously, no hotspots form in the upstream catalyst layer.

The activity advantageously increases continuously from the most inactive catalyst layer in flow direction.

In a preferred embodiment of a four-layer catalyst system including preliminary layer for the preparation of phthalic anhydride,
a) the upstream catalyst (preliminary layer) on nonporous and/or porous support material has from 7 to 11% by weight, based on the overall catalyst, of active composition, comprising from 4 to 11% by weight of V₂O₅, from 0 to 4% by weight of Sb₂O₃ or Nb₂O₅, from 0 to 0.5% by weight of P, from 0.1 to 0.8% by weight of alkali metal and, as the remainder, TiO₂ in anatase form,
b) the least active catalyst on nonporous and/or porous support material has from 7 to 11% by weight, based on the overall catalyst, of active composition, comprising from 4 to 11% by weight of V₂O₅, from 0 to 4% by weight of Sb₂O₃ or Nb₂O₅, from 0 to 0.5% by weight of P, from 0.1 to 1.1% by weight of alkali metal and, as the remainder, TiO₂ in anatase form,
c) the catalyst arranged next in flow direction on nonporous and/or porous support material has from 7 to 12% by weight, based on the overall catalyst, of active composition, comprising from 5 to 13% by weight of V₂O₅, 0 to 4% by weight of Sb₂O₃ or Nb₂O₅, from 0 to 0.5% by weight of P, from 0 to 0.4% by weight of alkali metal and, as the remainder, TiO₂ in anatase form,
d) and the catalyst arranged next in flow direction on nonporous and/or porous support has from 8 to 12% by weight, based on the overall catalyst, of active composition, comprising from 10 to 30% by weight of V₂O₅, from 0 to 4% by weight of Sb₂O₃ or Nb₂O₅, from 0 to 0.5% by weight of P, from 0 to 0.1% by weight of alkali metal and, as the remainder, TiO₂ in anatase form,
   the alkali metal used preferably being cesium.

The titanium dioxide used in anatase form advantageously has a BET surface area of from 5 to 50 m²/g, in particular from 15 to 40 m²/g. It is also possible to use mixtures of titanium dioxide in anatase form with different BET surface area, with the proviso that the resulting BET surface area has a value of from 15 to 40 m²/g. The individual catalyst layers may also comprise titanium dioxide with different BET surface areas. The BET surface area of the titanium dioxide used preferably increases from catalyst layer b) to catalyst layer d).

The activity of the catalyst layers advantageously increases from layer b) to layer d).

In a preferred embodiment of a five-layer catalyst system including preliminary layer,
a) the upstream catalyst (preliminary layer) on nonporous and/or porous support material has from 7 to 11% by weight, based on the overall catalyst, of active composition, comprising from 4 to 11% by weight of V₂O₅, from 0 to 4% by weight of Sb₂O₃ or Nb₂O₅, from 0 to 0.5% by weight of P, from 0.1 to 0.8% by weight of alkali metal and, as the remainder, TiO₂ in anatase form,
b) the least active catalyst on nonporous and/or porous support material has from 7 to 11% by weight, based on the overall catalyst, of active composition, comprising from 4 to 11% by weight of V₂O₅, from 0 to 4% by weight of Sb₂O₃ or Nb₂O₅, from 0 to 0.5% by weight of P, from 0.1 to 1.1% by weight of alkali metal and, as the remainder, TiO₂ in anatase form,
c1) the catalyst arranged next in flow direction on nonporous and/or porous support material has from 7 to 12% by weight, based on the overall catalyst, of active composition, comprising from 4 to 15% by weight of V₂O₅, 0 to 4% by weight of Sb₂O₃ or Nb₂O₅, from 0 to 0.5% by weight of P, from 0.1 to 1% by weight of alkali metal and, as the remainder, TiO₂ in anatase form,
c2) the catalyst arranged next in flow direction on nonporous and/or porous support material has from 7 to 12% by weight, based on the overall catalyst, of active composition, comprising from 5 to 13% by weight of V₂O₅, 0 to 4% by weight of Sb₂O₃ or Nb₂O₅, from 0 to 0.5% by weight of P, from 0 to 0.4% by weight of alkali metal and, as the remainder, TiO₂ in anatase form,
d) and the catalyst arranged next in flow direction on nonporous and/or porous support material has from 8 to 12% by weight, based on the overall catalyst, of active composition, comprising from 10 to 30% by weight of V₂O₅, 0 to 4% by weight of Sb₂O₃ or Nb₂O₅, from 0 to 0.5% by weight of P, from 0 to 0.1% by weight of alkali metal and, as the remainder, TiO₂ in anatase form,
   the alkali metal used preferably being cesium.

In general, the catalyst layers, for example b), c1), c2) and/or d), can also be arranged such that they each consist of two or more layers. These intermediate layers advantageously have intermediate catalyst compositions.

Instead of mutually delimited layers of the different catalysts, a quasi-continuous transition of the layers and a quasi-uniform rise in the activity can be brought about by providing a zone with a mixture of the successive catalysts at the transition from one layer to the next layer.

For the reaction, the catalysts are charged layer by layer into the tubes of a tube bundle reactor. The catalysts of different activity can be thermostatted to the same temperature or to different temperatures.

The present invention further relates to a process for gas phase oxidation in which a gaseous stream which comprises at least one hydrocarbon and molecular oxygen is passed through at least three catalyst layers arranged one on top of the other in a reaction tube, the least active catalyst layer being upstream of at least one more active catalyst layer.

The process according to the invention is advantageously suitable for the gas phase oxidation of aromatic C₆- to C₁₀-hydrocarbons such as benzene, the xylenes, toluene, naphthalene or durene (1,2,4,5-tetramethylbenzene) to carboxylic acids and/or carboxylic anhydrides such as maleic anhydride, phthalic anhydride, benzoic acid and/or pyromellitic anhydride.

The process is particularly suitable for preparing phthalic anhydride from o-xylene and/or naphthalene. The gas phase reactions for preparing phthalic anhydride are common knowledge and are described, for example, in WO 2004/103561 on page 6.

The present invention provides a catalyst system whose initial hotstop forms very close to the reactor inlet. As a result of the greater utilization of the catalyst bed toward the reactor inlet, longer lifetimes can be achieved. In addition, the undesired side reactions mentioned occur only at a later time than in the case of prior art catalyst systems as a result of the migration of the hotspot into more active catalyst layers.

### EXAMPLES

### Preparation of the Catalysts

### Catalyst VL1 (Preliminary Layer)

After stirring for 18 hours, 228.5 g of a suspension consisting of 104.9 g of oxalic acid, 39.4 g of vanadium pentoxide, 17.0 g of antimony oxide, 2.73 g of cesium sulfate, 2.95 g of ammonium dihydrogenphosphate, 149 g of formamide, 466.3 g of titanium dioxide and 720.0 g of water at 160° C., together with 12.5 g of organic binder, were applied in a coating drum to 1400 g of steatite rings of dimensions 8×6×5 mm (outer diameter×height×inner diameter). In a second step, the rings thus coated were coated with 236.9 g of a second suspension which had likewise been stirred beforehand for 18 h, consisting of 56.7 g of oxalic acid, 21.0 g of vanadium pentoxide, 2.73 g of cesium sulfate, 198 g of formamide, 502.1 g of titanium dioxide and 720.3 g of water together with 12.7 g of organic binder.

After the catalyst had been calcined at 450° C. for one hour, the active composition applied to the steatite rings was 9.7% The analyzed composition of the active composition consisted of 5.75% V₂O₅, 1.6% Sb₂O₃, 0.38% Cs, 0.08% P, remainder TiO₂.

### Catalyst HL1 (1st Main Layer)

Preparation analogous to VL1 with variation of the composition of the suspension. After the catalyst had been calcined at 450° C. for one hour, the active composition applied to the steatite rings was 9.2%. The analyzed composition of the active composition consisted of 5.81% V₂O₅, 1.64% Sb₂O₃, 0.44% Cs, 0.11% P, remainder TiO₂.

### Catalyst HL2 (2nd Main Layer)

Preparation analogous to VL1 with variation of the composition of the suspension. After the catalyst had been calcined at 450° C. for one hour, the active composition applied to the steatite rings was 9.3%. The analyzed composition of the active composition consisted of 5.66% V₂O₅, 1.58% Sb₂O₃, 0.18% Cs, 0.10% P, remainder TiO₂.

### Catalyst HL3 (3rd Main Layer)

Preparation analogous to VL1 with variation of the composition of the first suspension. A second coating is not performed. After the catalyst had been calcined at 450° C. for one hour, the active composition applied to the steatite rings was 9.9%. The analyzed composition of the active composition consisted of 7.42% V₂O₅, 3.2% Sb₂O₃, 0.07% Cs, 0.17% P, remainder TiO₂.

### Axial Composition of the Catalyst System

### A) Noninventive

The catalysts were introduced into a reaction tube of internal diameter 25 mm. Starting from the reactor inlet, the catalyst bed had the following composition:

### VL1/HL1/HL2/HL3=0/180/90/60 cm.

### B) Inventive

The catalysts were introduced into a reaction tube of internal diameter 25 mm. Starting from the reactor inlet, the catalyst bed had the following composition:

### VL1/HL1/HL2/HL3=45/135/90/60 cm.

### Catalytic Results

At the same volume flow rate (4 m³ (STP)/h), after running-up to 80 g/m³ (STP), the following results were achieved:

| | Run | Salt bath | Hotspot | Hotspot position | o-xylene | |
|---|---|---|---|---|---|---|
| | time in | temperature | temperature | from reactor | loading in | PA yield, |
| Catalyst | days | in ° C. | in ° C. | inlet in cm | g/m³ (STP) | in m/m % |
| A (not | 22 | 356 | 440 | 90 | 80.5 | 114.3 |
| inventive) | | | | | | |
| B (inventive) | 24 | 355 | 442 | 75 | 80.1 | 114.4 |

1. A catalyst system for preparing carboxylic acids and/or carboxylic anhydrides which has at least three catalyst layers arranged one on top of the other in the reaction tube, with the proviso that the most inactive catalyst layer is preceded in the upstream direction by a more active catalyst layer.

2. The catalyst system according to claim 1, wherein the upstream catalyst layer makes up from 5 to 25 percent of the overall catalyst bed.

3. The catalyst system according to claim 1, wherein no hotspots form in the upstream catalyst layer.

4. The catalyst system according to claim 1, wherein the higher activity of the upstream catalyst layer is established by virtue of a lower content of cesium, by virtue of a higher active mass per unit tube volume, by virtue of a higher content of vanadium, by virtue of a higher BET surface area or by virtue of a combination of these means.

5. The catalyst system according to claim 4, wherein the higher activity of the upstream catalyst layer is established by virtue of a content of cesium lower by from 5 to 25%, and/or by virtue of the use of from 110 to 150% of active composition, or by virtue of the use of from 110 to 150% of vanadium and/or by virtue of a BET surface area higher by from 10 to 50%, based on the downstream catalyst layer.

6. The catalyst system according to claim 1, wherein, in a four-layer catalyst system, the upstream catalyst layer has from 5 to 25%, the second catalyst layer from 25 to 60%, the third catalyst layer from 10 to 40% and the fourth catalyst layer from 10 to 40%, based on the total length of the catalyst bed.

7. The catalyst system according to claim 1, wherein, in a five-layer catalyst system, the upstream catalyst layer has from 5 to 25%, the second catalyst layer from 25 to 60%, the third catalyst layer from 5 to 30%, the fourth catalyst layer from 5 to 30%, the fifth catalyst layer from 5 to 30%, based on the total length of the catalyst bed.

8. The catalyst system according to claim 1 which has four catalyst layers arranged one on top of the other,
a) the upstream catalyst on nonporous and/or porous support material has from 7 to 11% by weight, based on the overall catalyst, of active composition, comprising from 4 to 11% by weight of V₂O₅, from 0 to 4% by weight of Sb₂O₃ or Nb₂O₅, from 0 to 0.5% by weight of P, from 0.1 to 0.8% by weight of alkali metal and, as the remainder, TiO₂ in anatase form,
b) the least active catalyst on nonporous and/or porous support material has from 7 to 11% by weight, based on the overall catalyst, of active composition, comprising from 4 to 11% by weight of V₂O₅, from 0 to 4% by weight of Sb₂O₃ or Nb₂O₅, from 0 to 0.5% by weight of P, from 0.1 to 1.1% by weight of alkali metal and, as the remainder, TiO₂ in anatase form,
c) the catalyst arranged next in flow direction on nonporous and/or porous support material has from 7 to 12% by weight, based on the overall catalyst, of active composition, comprising from 5 to 13% by weight of V₂O₅, 0 to 4% by weight of Sb₂O₃ or Nb₂O₅, from 0 to 0.5% by weight of P, from 0 to 0.4% by weight of alkali metal and, as the remainder, TiO₂ in anatase form,
d) and the catalyst arranged next in flow direction on nonporous and/or porous support material has from 8 to 12% by weight, based on the overall catalyst, of active composition, comprising from 10 to 30% by weight of V₂O₅, from 0 to 4% by weight of Sb₂O₃ or Nb₂O₅, from 0 to 0.5% by weight of P, from 0 to 0.1% by weight of alkali metal and, as the remainder, TiO₂ in anatase form.

9. The catalyst system according to claim 1 which has five catalyst layers arranged one on top of the other,
a) the upstream catalyst on nonporous and/or porous support material has from 7 to 11% by weight, based on the overall catalyst, of active composition, comprising from 4 to 11% by weight of V₂O₅, from 0 to 4% by weight of Sb₂O₃ or Nb₂O₅, from 0 to 0.5% by weight of P, from 0.1 to 0.8% by weight of alkali metal and, as the remainder, TiO₂ in anatase form,
b) the least active catalyst on nonporous and/or porous support material has from 7 to 11% by weight, based on the overall catalyst, of active composition, comprising from 4 to 11% by weight of V₂O₅, from 0 to 4% by weight of Sb₂O₃ or Nb₂O₅, from 0 to 0.5% by weight of P, from 0.1 to 1.1% by weight of alkali metal and, as the remainder, TiO₂ in anatase form,
c1) the catalyst arranged next in flow direction on nonporous and/or porous support material has from 7 to 12% by weight, based on the overall catalyst, of active composition, comprising from 4 to 15% by weight of V₂O₅, 0 to 4% by weight of Sb₂O₃ or Nb₂O₅, from 0 to 0.5% by weight of P, from 0.1 to 1% by weight of alkali metal and, as the remainder, TiO₂ in anatase form,
c2) the catalyst arranged next in flow direction on nonporous and/or porous support material has from 7 to 12% by weight, based on the overall catalyst, of active composition, comprising from 5 to 13% by weight of V₂O₅, 0 to 4% by weight of Sb₂O₃ or Nb₂O₅, from 0 to 0.5% by weight of P, from 0 to 0.4% by weight of alkali metal and, as the remainder, TiO₂ in anatase form,
d) and the catalyst arranged next in flow direction on nonporous and/or porous support material has from 8 to 12% by weight, based on the overall catalyst, of active composition, comprising from 10 to 30% by weight of V₂O₅, 0 to 4% by weight of Sb₂O₃ or Nb₂O₅, from 0 to 0.5% by weight of P, from 0 to 0.1% by weight of alkali metal and, as the remainder, TiO₂ in anatase form.

10. The catalyst system according to claim 8, wherein the activity of the catalysts increases from catalyst layer b) to catalyst layer d).

11. A process for gas phase oxidation in which a gaseous stream which comprises at least one hydrocarbon and molecular oxygen is passed through at least three catalyst layers arranged one on top of the other in a reaction tube, the least active catalyst layer being upstream of at least one more active catalyst layer.

12. The process according to claim 11 for preparing phthalic anhydride by catalytic gas phase oxidation of xylene and/or naphthalene with a molecular oxygen-comprising gas.

13. The catalyst system according to claim 2, wherein no hotspots form in the upstream catalyst layer.

14. The catalyst system according to claim 2, wherein the higher activity of the upstream catalyst layer is established by virtue of a lower content of cesium, by virtue of a higher active mass per unit tube volume, by virtue of a higher content of vanadium, by virtue of a higher BET surface area or by virtue of a combination of these means.

15. The catalyst system according to claim 3, wherein the higher activity of the upstream catalyst layer is established by virtue of a lower content of cesium, by virtue of a higher active mass per unit tube volume, by virtue of a higher content of vanadium, by virtue of a higher BET surface area or by virtue of a combination of these means.

16. The catalyst system according to claim 2, wherein, in a four-layer catalyst system, the upstream catalyst layer has from 5 to 25%, the second catalyst layer from 25 to 60%, the third catalyst layer from 10 to 40% and the fourth catalyst layer from 10 to 40%, based on the total length of the catalyst bed.

17. The catalyst system according to claim 3, wherein, in a four-layer catalyst system, the upstream catalyst layer has from 5 to 25%, the second catalyst layer from 25 to 60%, the third catalyst layer from 10 to 40% and the fourth catalyst layer from 10 to 40%, based on the total length of the catalyst bed.

18. The catalyst system according to claim 4, wherein, in a four-layer catalyst system, the upstream catalyst layer has from 5 to 25%, the second catalyst layer from 25 to 60%, the third catalyst layer from 10 to 40% and the fourth catalyst layer from 10 to 40%, based on the total length of the catalyst bed.

19. The catalyst system according to claim 5, wherein, in a four-layer catalyst system, the upstream catalyst layer has from 5 to 25%, the second catalyst layer from 25 to 60%, the third catalyst layer from 10 to 40% and the fourth catalyst layer from 10 to 40%, based on the total length of the catalyst bed.

20. The catalyst system according to claim 2, wherein, in a five-layer catalyst system, the upstream catalyst layer has from 5 to 25%, the second catalyst layer from 25 to 60%, the third catalyst layer from 5 to 30%, the fourth catalyst layer from 5 to 30%, the fifth catalyst layer from 5 to 30%, based on the total length of the catalyst bed.
